SEQUENCE LISTING (58 sequences)

SEQ ID NO: 1 - protein, 312 aa, Homo sapiens
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 2 - protein, 192 aa, Homo sapiens
MANNDAVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 3 - protein, 187 aa, Homo sapiens
AVLKRLEQKGAEADQIIEYLKQQVSLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 4 - protein, 163 aa, Homo sapiens
SLLKEKAILQATLREEKKLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 5 - protein, 146 aa, Homo sapiens
KLRVENAKLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 6 - protein, 139 aa, Homo sapiens
KLKKEIEELKQELIQAEIQNGVKQIAFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 7 - protein, 92 aa, Homo sapiens
AVTTVSSGTKEQIKGGTGDEKKAKEKIEKKGEKKEKKQQSIAGSADSKPIDVSRLDLRIGCIITARKHPDADSLYVEEVDVGEIAPRTVVSG

SEQ ID NO: 8 - protein, 80 aa, Homo sapiens
AVTTVSSGTKEQIKGGTGDEKKAKEKIEKKGEKKEKKQQSIAGSADSKPIDVSRLDLRIGCIITARKHPDADSLYVEEVD

SEQ ID NO: 9 - protein, 70 aa, Homo sapiens
AVTTVSSGTKEQIKGGTGDEKKAKEKIEKKGEKKEKKQQSIAGSADSKPIDVSRLDLRIGCIITARKHPD

SEQ ID NO: 10 - protein, 70 aa, Homo sapiens
AVTTVSSGTKEQIKGGTGDEKKAKEKIEKKGEKKEKKQQSIAGSADSKPIDVSRLDLRIGSIITARKHPD

SEQ ID NO: 11 - protein, 70 aa, Homo sapiens
AVTAVSSGTKEQIKGGTGDEKKAKEKIEKKGEKKEKKQQSIAGSADSKPIDVSRLDLRIGCIITARKHPD

SEQ ID NO: 12 - protein, 70 aa, Homo sapiens
AVTTVSSGTKEQIKGGAGDEKKAKEKIEKKGEKKEKKQQSIAGSADSKPIDVSRLDLRIGCIITARKHPD

SEQ ID NO: 13 - protein, 70 aa, Homo sapiens
AVTAVSSGTKEQIKGGTGDEKKAKEKIEKKGEKKEKKQQSIAGSADSKPIDVSRLDLRIGSIITARKHPD

SEQ ID NO: 14 - protein, 70 aa, Homo sapiens
AVTTVSSGTKEQIKGGAGDEKKAKEKIEKKGEKKEKKQQSIAGSADSKPIDVSRLDLRIGSIITARKHPD

SEQ ID NO: 15 - DNA, 35 bp, Artificial Sequence
  AIMP1-(1-192)-F
cggaattcatggcaaataatgatgctgttctgaag

SEQ ID NO: 16 - DNA, 32 bp, Artificial Sequence
  AIMP1-(1-192)-R
gtctcgagttagccactgacaactgtccttgg

SEQ ID NO: 17 - DNA, 32 bp, Artificial Sequence
  AIMP1-(6-192)-F
cggaattcgctgttctgaagagactggagcag

SEQ ID NO: 18 - DNA, 32 bp, Artificial Sequence; identical to SEQ ID NO: 16
  AIMP1-(6-192)-R

SEQ ID NO: 19 - DNA, 35 bp, Artificial Sequence
  AIMP1-(30-192)-F
cggaattctctctacttaaggagaaagcaattttg

SEQ ID NO: 20 - DNA, 32 bp, Artificial Sequence; identical to SEQ ID NO: 16
  AIMP1-(30-192)-R

SEQ ID NO: 21 - DNA, 35 bp, Artificial Sequence
  AIMP1-(47-192)-F
cggaattcaaacttcgagttgaaaatgctaaactg

SEQ ID NO: 22 - DNA, 32 bp, Artificial Sequence; identical to SEQ ID NO: 16
  AIMP1-(47-192)-R

SEQ ID NO: 23 - DNA, 35 bp, Artificial Sequence
  AIMP1-(54-192)-F
cggaattcaaactgaagaaagaaattgaagaactg

SEQ ID NO: 24 - DNA, 32 bp, Artificial Sequence; identical to SEQ ID NO: 16
  AIMP1-(54-192)-R

SEQ ID NO: 25 - DNA, 31 bp, Artificial Sequence
  AIMP1-(101-192)-F
cggaattcgcagtaacaaccgtatcttctgg

SEQ ID NO: 26 - DNA, 32 bp, Artificial Sequence; identical to SEQ ID NO: 16
  AIMP1-(101-192)-R

SEQ ID NO: 27 - DNA, 32 bp, Artificial Sequence
  AIMP1-(114-192)-F
cggaattcaaaggaggaacaggagacgaaaag

SEQ ID NO: 28 - DNA, 32 bp, Artificial Sequence; identical to SEQ ID NO: 16
  AIMP1-(114-192)-R

SEQ ID NO: 29 - DNA, 35 bp, Artificial Sequence; identical to SEQ ID NO: 15
  AIMP1-(1-46)-F

SEQ ID NO: 30 - DNA, 33 bp, Artificial Sequence
  AIMP1-(1-46)-R
gtctcgagttacttctcttccctcaaagttgcc

SEQ ID NO: 31 - DNA, 35 bp, Artificial Sequence; identical to SEQ ID NO: 15
  AIMP1-(1-53)-F

SEQ ID NO: 32 - DNA, 33 bp, Artificial Sequence
  AIMP1-(1-53)-R
gtctcgagttaagcattttcaactcgaagtttc

SEQ ID NO: 33 - DNA, 33 bp, Artificial Sequence
  AIMP1-(193-312)-F
cggaattcctggtgaatcatgttcctcttgaac

SEQ ID NO: 34 - DNA, 33 bp, Artificial Sequence
  AIMP1-(193-312)-R
gtctcgagttatttgattccactgttgctcatg

SEQ ID NO: 35 - DNA, 31 bp, Artificial Sequence; identical to SEQ ID NO: 25
  AIMP1-(101-180)-F

SEQ ID NO: 36 - DNA, 38 bp, Artificial Sequence
  AIMP1-(101-180)-R
gtctcgagttaatctacttcttccacatacaaagaatc

SEQ ID NO: 37 - DNA, 31 bp, Artificial Sequence; identical to SEQ ID NO: 25
  AIMP1-(101-170)-F

SEQ ID NO: 38 - DNA, 36 bp, Artificial Sequence
  AIMP1-(101-170)-R
gtctcgagttaatcagggtgttttctagcagttatg

SEQ ID NO: 39 - DNA, 31 bp, Artificial Sequence; identical to SEQ ID NO: 25
  AIMP1-(101-160)-F

SEQ ID NO: 40 - DNA, 34 bp, Artificial Sequence
  AIMP1-(101-160)-R
gtctcgagttaaccaattcgaagatccagacggg

SEQ ID NO: 41 - DNA, 31 bp, Artificial Sequence; identical to SEQ ID NO: 25
  AIMP1-(101-146)-F

SEQ ID NO: 42 - DNA, 35 bp, Artificial Sequence
  AIMP1-(101-146)-R
gtctcgagttagtcggcacttccagctattgattg

SEQ ID NO: 43 - DNA, 32 bp, Artificial Sequence
  AIMP1-(101-170)C161S-F
cgcatatggcagtaacaaccgtatcttctggt

SEQ ID NO: 44 - DNA, 65 bp, Artificial Sequence
  AIMP1-(101-170)C161S-R
cgctcgagttaatcagggtgttttctagcagttatgatgctaccaattcgaagatccagacggga

SEQ ID NO: 45 - DNA, 936 bp, Homo sapiens
atggcaaataatgatgctgttctgaagagactggagcagaagggtgcagaggcagatcaaatcattgaatatcttaagcagcaagtttctctacttaaggagaaagcaattttgcaggcaactttgagggaagagaagaaacttcgagttgaaaatgctaaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggcctggtgaatcatgttcctcttgaacagatgcaaaatcggatggtgattttactttgtaacctgaaacctgcaaagatgaggggagtattatctcaagcaatggtcatgtgtgctagttcaccagagaaaattgaaatcttggctcctccaaatgggtctgttcctggagacagaattacttttgatgctttcccaggagagcctgacaaggagctgaatcctaagaagaagatttgggagcagatccagcctgatcttcacactaatgatgagtgtgtggctacatacaaaggagttccctttgaggtgaaagggaagggagtatgtagggctcaaaccatgagcaacagtggaatcaaa

SEQ ID NO: 46 - DNA, 576 bp, Homo sapiens
atggcaaataatgatgctgttctgaagagactggagcagaagggtgcagaggcagatcaaatcattgaatatcttaagcagcaagtttctctacttaaggagaaagcaattttgcaggcaactttgagggaagagaagaaacttcgagttgaaaatgctaaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 47 - DNA, 561 bp, Homo sapiens
gctgttctgaagagactggagcagaagggtgcagaggcagatcaaatcattgaatatcttaagcagcaagtttctctacttaaggagaaagcaattttgcaggcaactttgagggaagagaagaaacttcgagttgaaaatgctaaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 48 - DNA, 489 bp, Homo sapiens
tctctacttaaggagaaagcaattttgcaggcaactttgagggaagagaagaaacttcgagttgaaaatgctaaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 49 - DNA, 438 bp, Homo sapiens
aaacttcgagttgaaaatgctaaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 50 - DNA, 417 bp, Homo sapiens
aaactgaagaaagaaattgaagaactgaaacaagagctaattcaggcagaaattcaaaatggagtgaagcaaataccatttccatctggtactccactgcacgctaattctatggtttctgaaaatgtgatacagtctacagcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 51 - DNA, 276 bp, Homo sapiens
gcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagatgtcggagaaatagccccaaggacagttgtcagtggc

SEQ ID NO: 52 - DNA, 240 bp, Homo sapiens
gcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgatgcagattctttgtatgtggaagaagtagat

SEQ ID NO: 53 - DNA, 210 bp, Homo sapiens
gcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgat

SEQ ID NO: 54 - DNA, 210 bp, Homo sapiens
gcagtaacaaccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggtagcatcataactgctagaaaacaccctgat

SEQ ID NO: 55 - DNA, 210 bp, Homo sapiens
gcagtaacagccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgat

SEQ ID NO: 56 - DNA, 210 bp, Homo sapiens
gcagtaacaaccgtatcttctggtaccaaagaacagataaaaggaggagcaggagacgaaaagaaagcgaaagagaaaattgaaaagaaaggagagaagaaggagaaaaaacagcaatcaatagctggaagtgccgactctaagccaatagatgtttcccgtctggatcttcgaattggttgcatcataactgctagaaaacaccctgat

SEQ ID NO: 57 - DNA, 210 bp, Homo sapiens
gcagtaacagccgtatcttctggtaccaaagaacagataaaaggaggaacaggagacgaaaagaaagcgaaagagaaaattgaaaagaaaggagagaagaaggagaaaaaacagcaatcaatagctggaagtgccgactctaagccaatagatgtttcccgtctggatcttcgaattggtagcatcataactgctagaaaacaccctgat

SEQ ID NO: 58 - DNA, 210 bp, Homo sapiens
gcagtaacaaccgtatcttctggtaccaaagaacagataaaaggaggagcaggagacgaaaagaaagcgaaagagaaaattgaaaagaaaggagagaagaaggagaaaaaacagcaatcaatagctggaagtgccgactctaagccaatagatgtttcccgtctggatcttcgaattggtagcatcataactgctagaaaacaccctgat

(19) United States
(12) Patent Application Publication
(10) Pub. No.: US 2011/0124582 A1
(43) Pub. Date: May 26, 2011

(54) NOVEL POLYPEPTIDE HAVING ANTI-TUMOR ACTIVITY

(75) Inventors: Sunghoon Kim, Seoul (KR); Jung Min Han, Seoul (KR); Sang Gyu Park, Seoul (KR); Yoen Sook Lee, Kwangju (KR)

(73) Assignee: ATYR PHARMA, INC., San Diego, CA (US)

(21) Appl. No.: 12/854,843

(22) Filed: Aug. 11, 2010

Related U.S. Application Data
(63) Continuation of application No. 12/525,524, now abandoned.
(60) Provisional application No. 60/898,708, filed on Feb. 1, 2007.

(51) Int. Cl. A61K 38/17; C07K 14/435; C07H 21/04; C12N 15/63; C12N 1/21; A61P 35/00; C12N 5/00

(52) U.S. Cl. 514/21.3; 530/324; 536/23.1; 435/320.1; 435/252.1; 435/375

(57) ABSTRACT

The present invention relates to a novel polypeptide having anti-tumor activity through inducing apoptosis of endothelial cell and use thereof. More particularly, the present invention relates to a method for inducing apoptosis of endothelial cell, and for preventing or treating cancer, comprising administering to a subject in need thereof an effective amount of (a) an isolated polypeptide having the amino acid sequence of SEQ ID NO: 9 or the amino acid sequence having at least 90% sequence homology to the amino acid sequence of SEQ ID NO: 9; or (b) an isolated polynucleotide encoding the polypeptide of (a).

### TECHNICAL FIELD

The present invention relates to a novel polypeptide having anti-tumor activity, and more particularly, to a novel polypeptide having anti-tumor activity through inducing apoptosis of endothelial cell.

### BACKGROUND ART

Various types of cells which form living organisms can die through various processes. Apoptosis, which is first described by Kerr et al. in 1972, is a physiological phenomenon that is necessary for maintaining homeostasis and developing normal organs in multicellular organisms. It is an intracellular mechanism which happens selectively in cells responding to a certain stimulus. Particularly, cells can induce programmed cell death by various stress such as starvation, virus, oxygen radicals or chromosome damaging agents. The apoptosis can be found by observing chromosomal DNA fragmentation, activation of caspase family and specific morphological changes such as chromatin condensation, blebbing, cell shrinkage and apoptotic body (John D R et al, 2000, J. Structur. Biol. 129, 346˜358; Takeuchi M et al, 1999, Apoptosis, 4, 461˜468).

It is known that the apoptosis not only performs a physiological function for maintaining homeostasis in tissue, but induces various diseases through cell proliferation or cell loss caused by abnormally inhibiting or activating of apoptosis. Also, apoptosis plays an important role in maintaining lymphocyte homeostasis, which is one of mechanisms for regulation of immune response, and killing of target cells by lymphocytes.

Recently, since apoptosis plays an important role in maintaining tissue homeostasis and proliferation of cell, novel examples show that many diseases including immune diseases and tumor are caused by abnormal regulation of apoptosis. Therefore, understanding of mechanisms for regulation of apoptosis becomes an important issue in order to understand mechanisms of many human disease and develop treatment protocols thereof. And apoptosis becomes a higher value-added part in Medicinal Pharmaceutical industry.

Meanwhile, AIMP1 (ARS-interacting multi-functional protein 1) was previously known as the p43 protein and renamed by the present inventors (Kim S. H. et al., Trends in Biochemical Sciences, 30:569-574, 2005). The AIMP1 is a protein consisting of 312 amino acids, which binds to a multi-tRNA synthetase complex to increase the catalytic activity of the multi-tRNA synthetase complex. The AIMP1 is highly expressed in microneuron in the resions of autoimmune diseases including encephalomyelitis, neuritis and uveitis in vitro. This phenomenon where the AIMP1 is highly expressed in a certain development stage and tissue suggests that the AIMP1 is related to inflammatory responses and cell apoptosis (Berger, A. C. et al., J. Immunother. 23:519-527, 2000). The present inventors have previously found that the AIMP1 and its N-terminal fragment can be used as effective cytokine, anti-tumor agents and angiogenesis inhibitors (Park H. et al., J. Leukoc. Biol., 71:223-230, 2002; Park S. G. et al., J. Biol. Chem., 277:45243-45248, 2002; Park H. et al., Cytokine, 21:148-53, 2002).

Meanwhile, a peptide consisting of numerous amino acids has shortcomings in that it is metabolized upon in vivo administration, leading to the cleavage of the peptide bond, and tends to decompose in a process of formulation. Thus, it is generally preferable to keep the length of peptides as short as possible for use as drugs. However, because the pharmacological activity of peptides needs to be kept, it is an important in the development of drugs to find the minimum length peptide(s) with activity comparable to that of a long-chain peptide.

Accordingly, during the development of a novel anti-tumor agent, the present inventors have found that a polypeptide including a part of amino acid sequences in the middle region of known AIMP1 protein, has anti-tumor activity by inducing apoptosis of endothelial cell, thereby completing the present invention.

### DISCLOSURE

### Technical Problem

Therefore, it is an object of the present invention to provide an isolated polypeptide comprising the amino acid sequence of SEQ ID NO: 9 or the amino acid sequence having at least 90% sequence homology to the amino acid sequence of SEQ ID NO: 9, or an isolated polynucleotide encoding the polypeptide and use for anti-tumor activity thereof.

### Technical Solution

To achieve the above object, the present invention provides an isolated polypeptide comprising the amino acid sequence of SEQ ID NO: 9 or the amino acid sequence having at least 90% sequence homology to the amino acid sequence of SEQ ID NO: 9, and an isolated polynucleotide encoding the polypeptide.

In another aspect, the present invention provides a pharmaceutical composition comprising the polypeptide and the polynucleotide encoding the polypeptide.

In still another aspect, the present invention provides methods for inducing apoptosis of endothelial cell and for preventing or treating cancer, comprising administering to a subject in need thereof an effective amount of the polypeptide or the polynucleotide encoding the polypeptide.

In still another aspect, the present invention provides use of the polypeptide or the polynucleotide encoding the polypeptide, for preparation a pharmaceutical composition for treating cancer.

Hereinafter, the present invention will be described in detail.

### Definition

Unless otherwise stated, all technical and scientific terms used herein have the same meanings as commonly understood by those skilled in the art to which the present invention pertains.

As used herein, the term “effective amount” refers to an amount effective in inducing apoptosis of endothelial cell or showing anti-tumor activity in vivo or in vitro.

As used herein, the term “subject” means mammals, and particularly animals including human beings, or the cells or tissues of animals. The subject may be patients in need of treatment. Also, the cells may preferably be endothelial cell.

The polypeptide of the present invention includes 101-170 amino acid sequence of the AIMP1 protein. Meanwhile, AIMP1 (ARS-interacting multi-functional protein 1) was previously known as the p43 protein and renamed by the present inventors (Sang Gyu Park, et al., Trends in Biochemical Sciences, 30:569-574, 2005).

The AIMP1 is a protein consisting of 312 amino acids, which binds to a multi-tRNA synthetase complex to increase the catalytic activity of the multi-tRNA synthetase complex. It is known that the AIMP1 is secreted from various types of cells, including prostate cancer cells, immune cells and transgenic cells, and the secreted AIMP1 works on diverse target cells such as monocytes/macrophages, endothelial cells and fibroblast cells. The following three SNPs of the AIMP1 are known (see NCBI SNP database): substitution of 79th alanine (Ala) to proline (Pro) (SNP accession no. rs3133166); substitution of 104th threonine (Thr) to alanine (Ala) (SNP accession no. rs17036670); and substitution of 117th threonine (Thr) to alanine (Ala) (SNP accession no. rs2230255) in the amino acid sequence of the full-length AIMP1 (SEQ ID NO: 1).

The present inventors constructed a series of deletion fragments of the AIMP1 so as to determine a functional domain of AIMP1 related to endothelial cell death (see FIG. 1 and FIG. 2), and then examined activity of fragments in inducing apoptosis of endothelial cell death (see <example 2>). Consequently, it could be supposed that a region of amino acid 101-192 of the AIMP1 would be a domain having the activity of inducing apoptosis of endothelial cell (see FIG. 4 and FIG. 5).

In order to more particularly determine the functional domain of AIMP1 related to endothelial cell death, the region of the AIMP1-(101-192) was cleaved to prepare small fragments (see <example 3-1>) and the activities of these fragments in inducing apoptosis of endothelial cell were also examined (see from FIG. 8 to FIG. 10).

Also, in order to confirm whether AIMP1-(101-170) mutant can induce apoptosis of endothelial cell, the present inventors constructed AIMP1-(101-170) C161S peptide by inducing C161S point mutation in the AIMP1-(101-170) peptide (see <example 4-1>). As a result of comparing endothelial cell death activity of AIMP1 full length, AIMP1-(101-170) polypeptide and AIMP1-(101-170) mutant polypeptide, the present inventors found that the AIMP1-(101-170) C161S had an activity of inducing endothelial cell death in the absence of DTT (see FIG. 11).

Moreover, the present inventors examined whether AIMP1-(101-170) peptide had anti-tumor activity. As a result, the present inventors found that the AIMP1-(101-170) had more effective anti-tumor activity than the AIMP1 full length protein (see FIG. 14) without side effect of body weight loss (see FIG. 12 and FIG. 13).

Therefore, the present invention provides a polypeptide comprising the amino acid sequence of SEQ ID NO: 9 or the amino acid sequence having at least 90% sequence homology to the amino acid sequence of SEQ ID NO: 9.

Preferably, the inventive polypeptide may consist of, but not limited to, the amino acid sequence selected from the group consisting of SEQ ID NO: 4 to SEQ ID NO: 8 and SEQ ID NO: 10 to SEQ ID NO: 14. The amino acid sequence select from SEQ ID NO: 11 to SEQ ID NO: 14 is a known SNP of the amino acid sequence of SEQ ID NO: 9 or SEQ ID NO: 10. Most preferably, the inventive polypeptide may consist of the amino acid sequence of SEQ ID NO: 9 or SEQ ID NO: 10.

Also, the inventive polypeptide may include functional equivalents of the polypeptide having the amino acid sequence of SEQ ID NO: 9, and preferably functional equivalents of the polypeptide having the amino acid sequence of SEQ ID NO: 9, as well as salts thereof. More particularly, the term “functional equivalents” refers to polypeptide comprising the amino acid sequence having at least 80% amino acid sequence homology (i.e., identity), preferably at least 90%, and more preferably at least 95% for example, 80%, 81%, 82%, 83%, 84%, 85%, 86%, 87%, 88%, 89%, 90%, 91%, 92%, 93%, 94%, 95%, 96%, 97%, 98%, 99%, and 100% to the amino acid sequence of SEQ ID NO: 9 that exhibit substantially identical physiological activity to the polypeptide of SEQ ID NO: 9. The sequence identity or homology is defined herein as the percentage of amino acid residues in the candidate sequence that are identical with amino acid sequence of SEQ ID NO: 9, after aligning the sequences and introducing gaps, if necessary, to achieve the maximum percent sequence identity, and not considering any conservative substitutions (as described above) as part of the sequence identity. None of N-terminal, C-terminal, or internal extensions, deletions, or insertions into the amino acid sequence of SEQ ID NO: 9 shall be construed as affecting sequence identity or homology. Thus, sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a predetermined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) can be used in conjunction with the computer program. For example, the percent identity can be calculated as: the total number of identical matches multiplied by 100 and then divided by the sum of the length of the longer sequence within the matched span and the number of gaps introduced into the longer sequences in order to align the two sequences.

The scope of the functional equivalents as used herein also encompasses derivatives obtained by modifying a part of the chemical structure of the inventive polypeptide while maintaining the basic framework and the activity inducing endothelial cell death or inhibiting cancer cell proliferation. For example, this includes structural modifications for altering the stability, storage, volatility or solubility of the polypeptide.

The polypeptide according to the present invention can be prepared by a genetic engineering method using the expression of recombinant nucleic acid encoding the same. For this purpose, a DNA molecule encoding the AIMP1 or its fragment is first constructed according to any conventional method. The DNA molecule may synthesized by performing PCR using suitable primers. Alternatively, the DNA molecule may also be synthesized by a standard method known in the art, for example using an automatic DNA synthesizer (commercially available from Biosearch or Applied Biosystems). The constructed DNA molecule is inserted into a vector comprising at least one expression control sequence (ex: promoter, enhancer) that is operatively linked to the DNA sequence so as to control the expression of the DNA molecule, and host cells are transformed with the resulting recombinant expression vector. The transformed cells are cultured in a medium and condition suitable to express the DNA sequence, and a substantially pure polypeptide encoded by the DNA sequence is collected from the culture medium. The collection of the pure polypeptide may be performed using a method known in the art, for example, chromatography.

In this regard, the term “substantially pure polypeptide” means the inventive polypeptide that does not substantially contain any other proteins derived from host cells. For the genetic engineering method for synthesizing the inventive polypeptide, the reader may refer to the following literatures: Maniatis et al., Molecular Cloning: A Laboratory Manual, Cold Spring Harbor Laboratory 1982; Sambrook et al., supra; Gene Expression Technology, Method in Enzymology, Genetics and Molecular Biology, Method in Enzymology, Guthrie & Fink (eds.), Academic Press, San Diego, Calif. 1991; and Hitzeman et al., J. Biol. Chem., 255, 12073-12080 1990.

Alternatively, the inventive polypeptide can be chemically synthesized according to any technique known in the art (Creighton, Proteins: Structures and Molecular Principles, W. H. Freeman and Co., N.Y., 1983). Namely, the inventive polypeptide can be prepared by conventional step-wise liquid or solid phase synthesis, fragment condensation, F-MOC or T-BOC chemistry (Chemical Approaches to the Synthesis of Peptides and Proteins, Williams et al., Eds., CRC Press, Boca Raton Fla., 1997; A Practical Approach, Atherton & Sheppard, Eds., IRL Press, Oxford, England, 1989).

It is particularly preferred to use the solid phase synthesis to prepare the inventive peptide. The inventive polypeptide can be synthesized by performing the condensation reaction between protected amino acids by the conventional solid-phase method, beginning with the C-terminal and progressing sequentially with the first amino acid, the second amino acid, the third amino acid, and the like according to the identified sequence. After the condensation reaction, the protecting groups and the carrier connected with the C-terminal amino acid may be removed by a known method such as acid decomposition or aminolysis. The above-described peptide synthesis method is described in detail in the literature (Gross and Meienhofer's, The peptides, vol. 2, Academic Press, 1980). Examples of a solid-phase carrier, which can be used in the synthesis of the polypeptide according to the present invention, include polystyrene resins of substituted benzyl type, polystyrene resins of hydroxymethylphenylacetic amide form, substituted benzhydrylpolystyrene resins and polyacrylamide resins, having a functional group capable of bonding to peptides. Also, the condensation of amino acids can be performed using conventional methods, for example dicyclohexylcarbodimide (DDC) method, acid anhydride method and activated ester method.

Protecting groups used in the synthesis of the inventive peptide are those commonly used in peptide syntheses, including those readily removable by conventional methods such as acid decomposition, reduction or aminolysis. Specific examples of such amino protecting groups include formyl; trifluoroacetyl; benzyloxycarbonyl; substituted benzyloxycarbonyl such as (ortho- or para-) chlorobenzyloxycarbonyl and (ortho- or para-) bromobenzyloxycarbonyl; and aliphatic oxycarbonyl such as t-butoxycarbonyl and t-amiloxycarbonyl. The carboxyl groups of amino acids can be protected through conversion into ester groups. The ester groups include benzyl esters, substituted benzyl esters such as methoxybenzyl ester; alkyl esters such as cyclohexyl ester, cycloheptyl ester or t-butyl ester. The guanidino moiety may be protected by nitro; or arylsulfonyl such as tosyl, methoxybenzensulfonyl or mesitylenesulfonyl, even though it does not need a protecting group. The protecting groups of imidazole include tosy, benzyl and dinitrophenyl. The indole group of tryptophan may be protected by formyl or may not be protected. Deprotection and separation of protecting groups from carriers can be carried out using anhydrous hydrofluoride in the presence of various scavengers. Examples of the scavengers include those commonly used in peptide syntheses, such as anisole, (ortho-, meta- or para-) cresol, dimethylsulfide, thiocresol, ethanendiol and mercaptopyridine.

The recombinant peptide prepared by the genetic engineering method or the chemically synthesizing can be isolated and purified according to methods known in the art, including extraction, recrystallization, various chromatographic techniques (e.g., gel filtration, ion exchange, precipitation, adsorption, reverse phase, etc.), electrophoresis and counter current distribution.

Moreover, the present invention provides an isolated polynucleotide encoding the isolated polypeptide having the amino acid sequence of SEQ ID NO: 9 or the amino acid sequence having at least 90% sequence homology to the amino acid sequence. The polynucleotide comprises DNA, cDNA and RNA sequences. Preferably, the polynucleotide has the base sequence selected from the group consisting of SEQ ID NO: 48 to SEQ ID NO: 58. The polynucleotide of SEQ ID NO: 55 to SEQ ID NO: 58 encodes the known SNP of the polypeptide of SEQ ID NO: 9 or SEQ ID NO: 10. The polynucleotide can be prepared by separating from nature materials or genetic engineering methods known in the art to which the present invention pertains.

Also, the present invention provides a vector containing the polynucleotide according to the present invention. The vector may be, but not limited to, a plasmid or viral vector. The inventive polynucleotide can be introduced into a target cell by inserting it into the vector and then introducing the vector into a target cell by any method known in the art, such as infection, transfection and transduction.

Therefore, the present invention provides a transformant transformed with the vector. Particularly, a gene transfer method using a plasmid expression vector is a method of transferring a plasmid DNA directly to mammalian cells, which is an FDA-approved method applicable to human beings (Nabel, E. G., et al., Science, 249:1285-1288, 1990). Unlike viral vectors, the plasmid DNA has an advantage of being homogeneously purified. Plasmid expression vectors which can be used in the present invention include mammalian expression plasmids known in the pertinent art. For example, they are not limited to, but typically include pRK5 (European Patent No. 307,247), pSV16B (PCT Publication No. 91/08291) and pVL1392 (PharMingen). The plasmid expression vector containing the polynucleotide according to the present invention may be introduced into target cells by any method known in the art, including, but not limited to, transient transfection, microinjection, transduction, cell fusion, calcium phosphate precipitation, liposome-mediated transfection, DEAE dextran-mediated transfection, polybrene-mediated transfection, electroporation, gene gun methods, and other known methods for introducing DNA into cells (Wu et al., J. Bio. Chem., 267:963-967, 1992; Wu and Wu, J. Bio. Chem., 263:14621-14624, 1988).

In addition, virus expression vectors containing the polynucleotide according to the present invention include, but are not limited to, retrovirus, adenovirus, herpes virus, avipox virus and so on. The retroviral vector is so constructed that non-viral proteins can be produced within the infected cells by the viral vector in which virus genes are all removed or modified. The main advantages of the retroviral vector for gene therapy are that it transfers a large amount of genes into replicative cells, precisely integrates the transferred genes into cellular DNA, and does not induce continuous infections after gene transfection (Miller, A. D., Nature, 357:455-460, 1992). The retroviral vector approved by FDA was prepared using PA317 amphotropic retrovirus packaging cells (Miller, A. D. and Buttimore, C., Molec. Cell Biol., 6:2895-2902, 1986). Non-retroviral vectors include adenovirus as described above (Rosenfeld et al., Cell, 68:143-155, 1992; Jaffe et al., Nature Genetics, 1:372-378, 1992; Lemarchand et al., Proc. Natl. Acad. Sci. USA, 89:6482-6486, 1992). The main advantages of adenovirus are that it transfers a large amount of DNA fragments (36 kb genomes) and is capable of infecting non-replicative cells at a very high titer. Moreover, herpes virus may also be useful for human genetic therapy (Wolfe, J. H., et al., Nature Genetics, 1:379-384, 1992). The lentivirus is a kind of retrovirus and developed to new retroviral vector since the late 1990s. The lentiviral vector is constructed by modifying HIV backbone. It has high transfection efficiency in dividing and non-diving cells since it is not influenced by cell cycle unlike other retroviral vectors. Thus it has been developed as potential vectors for gene transfer in the cell therapy field using hematopoietic and keratinocyte stem cells, since transfection efficiency of the lentiviral vector in slow-dividing cell such as hematopoietic cell is higher than that of other viral vectors. Besides, other known suitable viral vectors can be used.

The transformation with the vector can be carried out according to any known transformation method in the pertinent art, preferably, microprojectile bombardment, electroporation, CaPO₄ precipitation, CaCl₂ precipitation, PEG-mediated fusion, microinjection and liposome-mediated method, but not limited to. The transformant may be Escherichia coli, Bacillus subtilis, Streptomyces, Pseudomonas, Proteus mirabilis and Staphylococcus, Agrobacterium tumefaciens, but not limited to.

Also, the present invention provides a pharmaceutical composition comprising the inventive polypeptide or polynucleotide encoding the same and a pharmaceutically acceptable salt. Preferably, the inventive pharmaceutical composition can be, but not limited to, the pharmaceutical composition for treating cancer.

The inventive pharmaceutical composition may further comprise a pharmaceutically acceptable carrier or excipient. The carrier or excipient can include, but not limited to, dispersing agents, wetting agents, suspension agents, diluents, and fillers. The ratio between the pharmaceutically acceptable carriers and expression vectors included in the inventive pharmaceutical composition, is fixed by solubility and chemical properties of the composition or administration ways.

The therapeutic or preventive effective amount of the inventive pharmaceutical composition containing the AIM1 protein-encoding polynucleotide may be suitably selected depending on the subject to be administered, age, individual variation and disease condition.

As used herein, the term “pharmaceutically acceptable” means what is physiologically acceptable and, when administered to human beings, generally does not cause allergic reactions, such as gastrointestinal disorder and dizziness, or similar reactions thereto.

Examples of the pharmaceutically acceptable salt include salts with inorganic bases, salts with organic bases, salts with inorganic acids, salts with organic acids, salts with basic or acidic amino acids and the like. Examples of the salt with an inorganic acid include alkali metal salts, such as a sodium salt and a potassium salt; an alkaline earth metal salt such as a calcium salt and a magnesium salt; an aluminum salt; and an ammonium salt. Examples of the salt with an organic base include salts with trimethylamine, triethylamine, pyridine, picoline, 2,6-lutidine, ethanolamine, diethanolamine, triethanolamine, cyclohexylamine, dicyclohexylamine and N,N′-dibenzylethylenediamine. Examples of the salt with an inorganic acid include salts with hydrochloric acid, boric acid, nitric acid, sulfuric acid and phosphoric acid. Examples of the salt with an organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid and p-toluenesulfonic acid. Examples of the salt with a basic amino acid include salts with arginine, lysine and ornithine. Examples of the salt with an acidic amino acid include salts with aspartic acid and glutamic acid. The list of suitable salts is disclosed in Remington's Pharmaceutical Sciences, 17th ed., Mack Publishing Company, Easton, Pa., p1418, 1985, the entire disclosure of which is incorporated herein by reference.

Also, the inventive pharmaceutical composition may also be formulated, but not limited to, as preparations for oral administration. For oral administration, the inventive polypeptide, the polynucleotide coding the same, and the pharmaceutically acceptable salt mixed with the excipients, can be formulated in the form of ingestible tablets, buccal tablets, troches, capsules, elixirs, suspensions, syrups, wafers, and the like. These preparations may also comprise diluents (e.g., lactose, dextrose, sucrose, mannitol, sorbitol, cellulose and/or glycine), lubricants (e.g., silica, talc, stearic acid and a magnesium or calcium salt thereof, and/or polyethylene glycol) in addition to the active ingredient. Among various preparations, tablets may also comprise binders, such as magnesium aluminum silicate, starch pastes, gelatin, tragacanth, methylcellulose, sodium carboxymethylcellulose and/or polyvinylpyrrolidone, and, if desired, may further comprise disintegrating agents, such as starches, agar or alginic acid or a sodium salt thereof, absorbents, colorants, flavors and sweeteners. These formulations may be prepared by a conventional mixing, granulation or coating method. The inventive pharmaceutical composition may be administered itself or in the form of various formulations as described above, and preferably it may be administered until the desired effect, i.e., effect of endothelial cell death and/or anti-tumor. The inventive pharmaceutical composition may be administered by various routes according to any method known in the art. Namely, it may be administered by oral or parenteral routes. For example, the parenteral routes include methods for applying to the skin locally, intramuscular, intravenous, intracutaneous, intraarterial, intramarrow, intrathecal, intraperitoneal, intranasal, intravaginal, intrarectal, sublingual and subcutaneous or administering to gastrointestinal tracts, mucosa or respiratory organs systemically. For example, The inventive pharmaceutical composition may be administered by a method of applying the polypeptide directly to the skin or a method comprising formulating the polypeptide in an injectable form, and then, injecting a given amount of the formulation into a subcutaneous layer with a 30-gauge injection needle or lightly pricking the skin with an injection needle. Preferably, the inventive polypeptide may be applied directly to the skin.

Also, The inventive pharmaceutical composition may also be administered in a form bound to a molecule causing a high-affinity binding to a target cell or tissue (e.g., skin cell or skin tissue) or in a form encapsulated in the molecule. The inventive pharmaceutical composition can be bound to sterol (e.g., cholesterol), a lipid (e.g., a cationic lipid, virosome or liposome), or a target cell-specific binding agent (e.g., a ligand recognized by target cell specific receptor) using the technology known in the art. Suitable coupling agents or crosslinking agents may include, for example, protein A, carbodiimide, and N-succinimidyl-3-(2-pyridyldithio) propionate (SPDP).

The total effective amount of the polypeptide in the inventive pharmaceutical composition can be administered to a subject as a single dose, or can be administered using a fractionated treatment protocol, in which the multiple doses are administered over a more prolonged period of time. The amount of the active ingredient in the composition containing the inventive polypeptide may vary depending on the use of the composition, but the active ingredient may be generally administered at an effective dose of 0.1 μg-1 g several times daily. However, the effective dose of the polypeptide may vary depending on many factors, such as the age, body weight, health condition, sex, disease severity, diet and excretion of a subject in need of treatment, as well as administration time and administration route. In view of these factors, any person skilled in the art may determine an effective dose suitable for the above-described specific use of the inventive polypeptide. The inventive composition has no special limitations on its formulation, administration route and administration mode as long as it shows the effects of the present invention.

In another aspect, the inventive polypeptide or polynucleotide encoding the polypeptide can be used in a method for inducing apoptosis of endothelial cell. Therefore, the present invention provides a method for inducing apoptosis of endothelial cell, comprising administering to a subject in need thereof an effective amount of the polypeptide or the polynucleotide encoding the polypeptide.

Also, the present invention provides a method for preventing or treating cancer, comprising administering to a subject in need thereof an effective amount of the polypeptide or the polynucleotide encoding the polypeptide.

The cancers include, but are not limited to, breast cancer, rectal cancer, lung cancer, small-cell lung cancer, stomach cancer, liver cancer, blood cancer, bone cancer, pancreatic cancer, skin cancer, head or neck cancer, skin or intraocular melanoma, uterine carcinoma, ovarian cancer, colorectal cancer, cancer near the anus, colon cancer, oviduct carcinoma, endometrial carcinoma, cervical cancer, vaginal cancer, vulva carcinoma, Hodgkin's disease, esophagus cancer, small intestinal tumor, endocrine gland cancer, thyroid cancer, parathyroid cancer, adrenal cancer, soft-tissue sarcoma, uterine cancer, penis cancer, prostate cancer, chronic or acute leukemia, lymphocytic lymphoma, bladder cancer, kidney or urethra cancer, kidney cell carcinoma, kidney pelvis carcinoma, CNS tumor, primary CNS lymphoma, spinal tumor, brain stem glioma, and pituitary adenoma, and a combination of one or more thereof.

In still another aspect, the present invention provides the polypeptide or the polynucleotide encoding the polypeptide for use as a medicament.

Also, the present invention provides use of the polypeptide or the polynucleotide encoding the polypeptide for preparation a pharmaceutical composition for treating cancer.

### ADVANTAGEOUS EFFECTS

The polypeptide or the polynucleotide encoding the polypeptide has the activity inhibiting cancer cell proliferation by inducing apoptosis of endothelial cell. Therefore, the polypeptide or the polynucleotide encoding the polypeptide can be effectively used in inducing apoptosis of endothelial cell, or preventing or treating cancer.

### DESCRIPTION OF DRAWINGS

FIG. 1 is a schematic drawing of the AIMP1 fragments of the present invention.

FIG. 2 shows the results of SDS-PAGE analysis for the AIMP1 fragments of the present invention.

FIG. 3 is a picture showing the activity of inducing cell death of the AIMP1 fragments of the present invention.

FIG. 4 is a graph showing the activity of inducing cell death of the AIMP1 fragments of the present invention.

FIG. 5 is a graph showing the effect of the AIMP1 fragments of the present invention for the activity of caspase-3.

FIG. 6 is a schematic drawing of the AIMP1-(101-192) fragments of the present invention.

FIG. 7 shows the result of electrophoresis for the AIMP1-(101-192) fragments of the present invention.

FIG. 8 is a picture showing the activity of inducing of cell death of the AIMP1-(101-192) fragments of the present invention.

FIG. 9 is a graph showing the measurement result for cell viability by MTT assay after the treatment of the AIMP1-(101-192) fragments of the present invention.

FIG. 10 is a graph showing the effect on the activity of caspase-3 of the AIMP1-(101-192) fragments of the present invention.

FIG. 11 is a graph showing analysis results for activity of endothelial cell apoptosis of the AIMP1-(101-170) C161S mutant polypeptide of the present invention.

FIG. 12 is a graph showing analysis results for tumor cell anti-proliferation activity of the AIMP1-(101-170) polypeptide of the present invention.

FIG. 13 is a graph showing analysis results for body weight loss after the treatment of the AIMP1-(101-170) polypeptide of the present invention.

FIG. 14 is a graph showing analysis results for tumor cell anti-proliferation activity of the AIMP1-(101-170) polypeptide of the present invention in comparison with AIMP1 full length protein.

FIG. 15 is a graph showing analysis results for body weight loss after the treatment of the AIMP1-(101-170) polypeptide of the present invention in comparison with AIMP1 full length protein.

### MODE FOR INVENTION

Reference will now be made in detail to the preferred embodiments of the present invention. It is to be understood that the following examples are illustrative only and the present invention is not limited thereto.

### Example 1

### Construction of AIMP1 Protein or its Fragments

An AIMP1 consisting of 312 amino acids (SEQ ID NO: 1) was constructed according to the method of Park et al. (Park S. G. et al., J. Biol. Chem., 277:45243-45248, 2002).

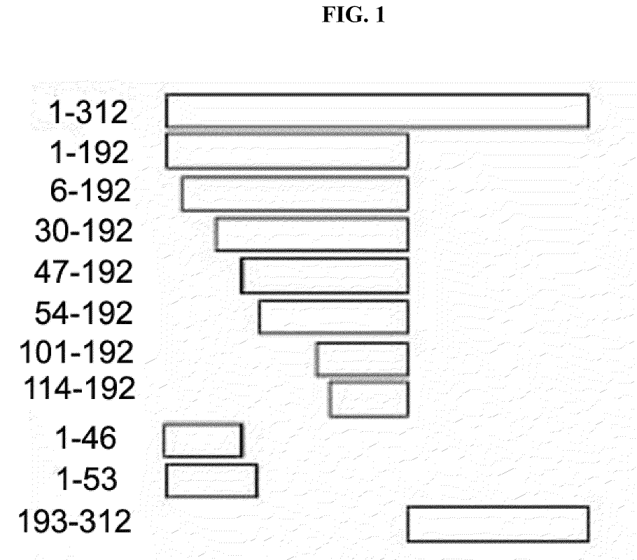

Also, Each of deletion fragments of AIMP1 shown in FIG. 1, i.e., AIMP1-(1-192) (SEQ ID NO: 2), AIMPI-(6-192) (SEQ ID NO: 3), AIMP1-(30-192) (SEQ ID NO: 4), AIMP1-(47-192) (SEQ ID NO: 5), AIMP1-(54-192) (SEQ ID NO: 6), AIMP1-(101-192) (SEQ ID NO: 7), AIMP1-(114-192), AIMP1-(1-46), AIMP1-(1-53) and AIMP1-(193-312) fragments was constructed. Each of the fragments was synthesized by PCR using the cDNA of AIMP1 (SEQ ID NO: 1) as a template with specific primer sets (see Table 1). The PCR reaction conditions were as follows: pre-denaturation of template DNA by heating at 95° C. for 2 min; and then 30 cycles at 95° C. for 30 sec, 56° C. for 30 sec and 72° C. for 1 min; followed by final extension at 72° C. for 5 min.

TABLE 1

| | | | SEQ. ID |
|---|---|---|---|
| Primer | | Sequence | NO |
| AIMP1- | sense | 5′-CGGAATTCAT GGCAAATAAT | 15 |
| (1-192) | | GATGCTGTTC TGAAG-3′ | |
| | anti- | 5′-GTCTCGAGTT AGCCACTGAC | 16 |
| | sense | AACTGTCCTT GG-3′ | |
| AIMP1- | sense | 5′-CGGAATTCGC TGTTCTGAAG | 17 |
| (6-192) | | AGACTGGAGC AG-3′ | |
| | anti- | 5′-GTCTCGAGTT AGCCACTGAC | 18 |
| | sense | AACTGTCCTT GG-3′ | |
| AIMP1- | sense | 5′-CGGAATTCTC TCTACTTAAG | 19 |
| (30-192) | | GAGAAAGCAA TTTTG-3′ | |
| | anti- | 5′-GTCTCGAGTT AGCCACTGAC | 20 |
| | sense | AACTGTCCTT GG-3′ | |
| AIMP1- | sense | 5′-CGGAATTCAA ACTTCGAGTT | 21 |
| (47-192) | | GAAAATGCTA AACTG-3′ | |
| | anti- | 5′-GTCTCGAGTT AGCCACTGAC | 22 |
| | sense | AACTGTCCTT GG-3′ | |
| AIMP1- | sense | 5′-CGGAATTCAA ACTGAAGAAA | 23 |
| (54-192) | | GAAATTGAAG AACTG-3′ | |
| | anti- | 5′-GTCTCGAGTT AGCCACTGAC | 24 |
| | sense | AACTGTCCTT GG-3′ | |
| AIMP1- | sense | 5′-CGGAATTCGC AGTAACAACC | 25 |
| (101-192) | | GTATCTTCTG G-3′ | |
| | anti- | 5′-GTCTCGAGTT AGCCACTGAC | 26 |
| | sense | AACTGTCCTT GG-3′ | |
| AIMP1- | sense | 5′-CGGAATTCAA AGGAGGAACA | 27 |
| (114-192) | | GGAGACGAAA AG-3′ | |
| | anti- | 5′-GTCTCGAGTT AGCCACTGAC | 28 |
| | sense | AACTGTCCTT GG-3′ | |
| AIMP1- | sense | 5′-CGGAATTCAT GGCAAATAAT | 29 |
| (1-46) | | GATGCTGTTC TGAAG-3′ | |
| | anti- | 5′-GTCTCGAGTT ACTTCTCTTC | 30 |
| | sense | CCTCAAAGTT GCC-3′ | |
| AIMP1- | sense | 5′-CGGAATTCAT GGCAAATAAT | 31 |
| (1-53) | | GATGCTGTTC TGAAG-3′ | |
| | anti- | 5′-GTCTCGAGTT AAGCATTTTC | 32 |
| | sense | AACTCGAAGT TTC-3′ | |
| AIMP1- | sense | 5′-CGGAATTCCT GGTGAATCAT | 33 |
| (193-312) | | GTTCCTCTTG AAC-3′ | |
| | anti- | 5′-GTCTCGAGTT ATTTGATTCC | 34 |
| | sense | ACTGTTGCTC ATG-3′ | |

Primer sets used for preparing AIMP1 fragments

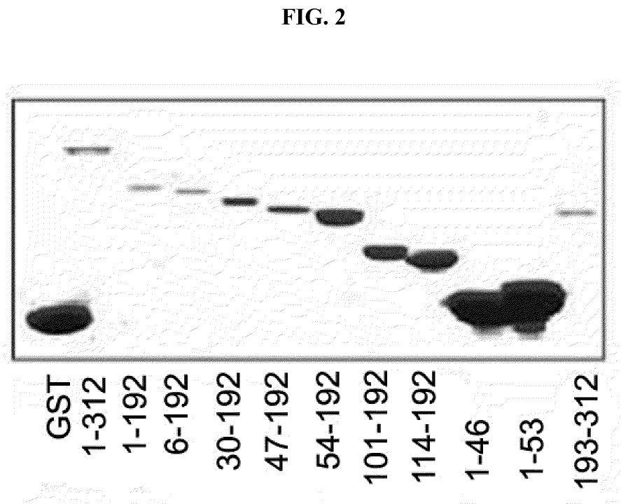

Each of the PCR products was digested with EcoRI and Xhol and ligated into a pGEX4T3 vector (Amersham Biosciences) digested with the same restriction enzymes. E.coli BL21 (DE3) was transformed with the vector and cultured to induce expression of the polypeptides. Each of the polypeptides was expressed as a GST-tag fusion protein and purified on GSH agarose gel. To remove lipopolysaccharide, protein solution was dialyzed through pyrogen-free buffer (10 mM potassium phosphate buffer, pH 6.0, 100 mM NaCl). After the dialysis, the solution was loaded onto polymyxin resin (Bio-Rd) pre-equilibrated with the same buffer and then incubated for 20 minutes followed by elution. Concentration of residual lipopolysaccharide (LPS) was below 20 pg/Ml when determined using a Limulus Amebocyte Lysate QCL-1000 kit. Each of the purified peptides was analyzed by SDS-PAGE and the result was shown in FIG. 2.

As a result shown in FIG. 2, AIMP1-(1-192) (SEQ ID NO: 2), AIMPI-(6-192) (SEQ ID NO: 3), AIMP1-(30-192) (SEQ ID NO: 4), AIMP1-(47-192) (SEQ ID NO: 5), AIMP1-(54-192) (SEQ ID NO: 6), AIMP1-(101-192) (SEQ ID NO: 7), AIMP1-(114-192), AIMP1-(1-46), AIMP1-(1-53) and AIMP1-(193-312) fragments could be constructed.

### Example 2

### Identification of AIMP1 Domain Having Activity of Inducing Cell Death

### <2-1> Identification of Fragments Having Activity of Inducing Apoptosis Endothelial Cell

To confirm whether the deletion fragments of the AIMP1 constructed in the <Example 1> had cell death-inducing activity, the present inventors investigated the effect on cell death by treating BAECs (Bovine aorta endotheilial cells) with the fragments of AIMP.

BAECs (Bovine aorta endothelial cells) were isolated from descending thoracic aortas and grown in Dulbecco's modified Eagle's medium containing 20% fetal bovine serum at 37° C. in a 5% CO₂ atmosphere. The cultured BAECs were treated with the deletion fragments of the AIMP1 (50 nM) for 24 h, and apoptotic cells were counted.

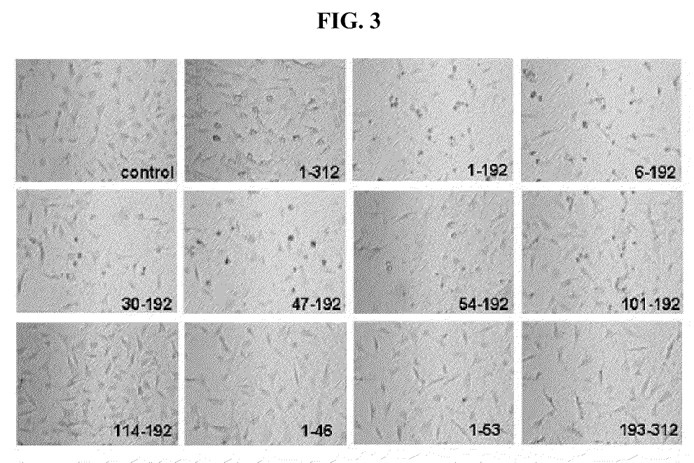

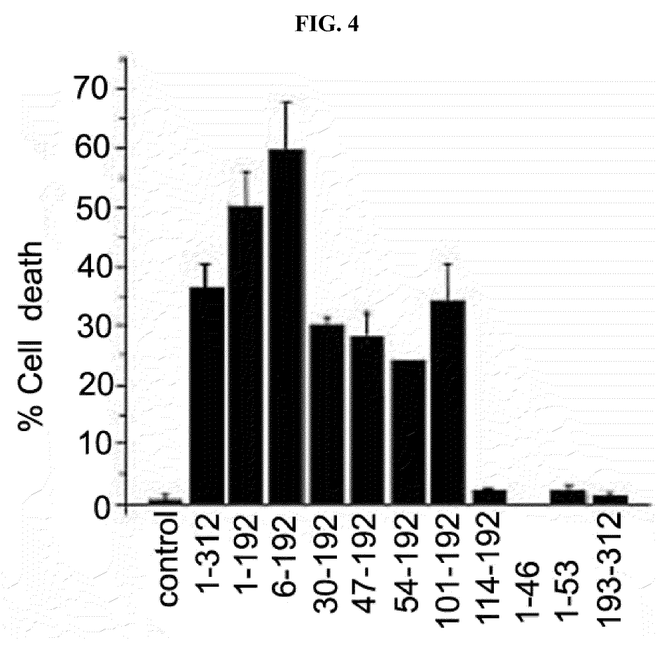

Concretely, enhanced green fluorescent protein (EGFP) were transfected into BAECs, and expressed for 24 h. The transfected cells were treated with the fragments of the AIMP1 (50 nM) for 24 h, and then cell death was determined by counting apoptotic cells using fluorescence microscopy. The percentage of apoptotic cells was determined by dividing the number of green cells with apoptotic morphology by the total number of green cells (see FIGS. 3 and 4).

As shown in FIG. 3 and FIG. 4, it was found that AIMP1-(1-312), AIMP1-(1-192), AIMP1-(6-192), AIMP1-(30-192), AIMP1-(47-192), AIMP1-(54-192), and AIMP1-(101-192) of the deletion fragments of the AIMP1 constructed in the <Example 1>, could induce apoptosis of endothelial cell at high levels, but AIMP1-(114-192), AIMP1-(1-46), AIMP1-(1-53), AIMP1-(30-192) and AIMP1-(193-312) could not induce apoptosis.

From the result, it was believed that the middle region of AIMP1, especially AIMP1-(101-192), might be a cell death-inducing domain on endothelial cells.

### <2-2> Measurement of Caspase-3 Activation

Since AIMP1 induced endothelial cell apoptosis through caspase-3 activation (S. G. Park, et. al., J. Biol. Chem., 277:4524345248, 2002), the present inventors reconfirmed whether the fragments of AIMP had cell death-inducing activity through measuring caspase-3 activity.

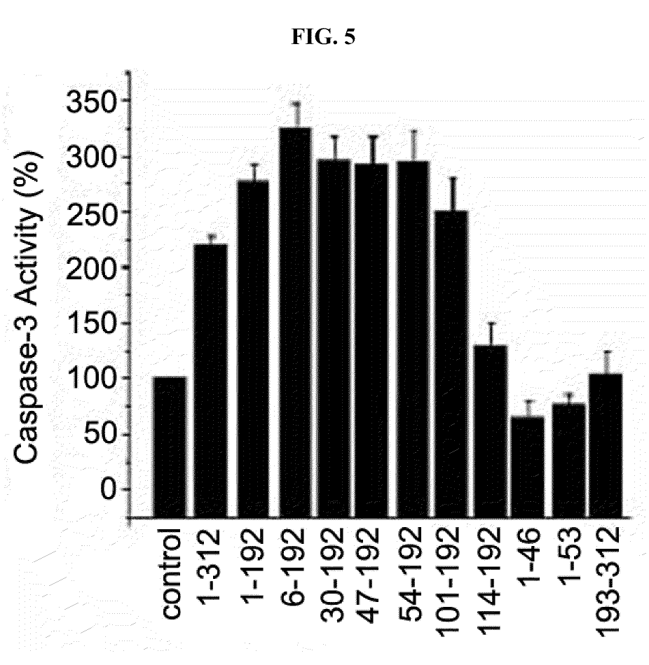

Concretely, BAECs (2×10⁶) were harvest and lysed with 300 μl of cell lysis buffer (20 mM HEPES, pH 7.5, 1 mM dithiothreitol (DTT), 0.1 mM EDTA, 0.5% NP-40, and 0.1 mM PMSF). The cell lysates were centrifuged at 15,000×g for 5 min and the supernatant fractions were used to measure caspase activity. Aliquots of 40 μl of cell lysate protein were incubated for 2 h at 30° C. in an assay buffer (20 mM HEPES at pH 7.5, 2 mM DTT, 10% glycerol) containing 100 uM Ac-DEVD-p-nitroanilide for a caspase-3 substrate or Ac-YVAD-p-nitroanilide for caspase-1 substrate. The amount of p-nitroaniline released by caspase activity was quantitated by measuring the optical density at 405 nm (see FIG. 5).

The result shown in FIG. 5 was similar to that shown in FIG. 3 and FIG. 4. From these results, it was believed that the middle region of AIMP1, especially AIMP1-(101-192) might be a cell death-inducing domain on endothelial cell.

### Example 3

### Construction of Additional Deletion Fragments of AIMP1-(101-192) and Measurement of Their Activity

### <3-1> Construction of Additional Deletion Fragments of AIMP1-(101-192)

In order to more particularly determine cell death-inducing domain of AIMP1-(101-192) estimated to be a cell death-inducing domain in endothelial cell from the results in the <Example 2>, the present inventors constructed additional deletion fragments of AIMP1-(101-192).

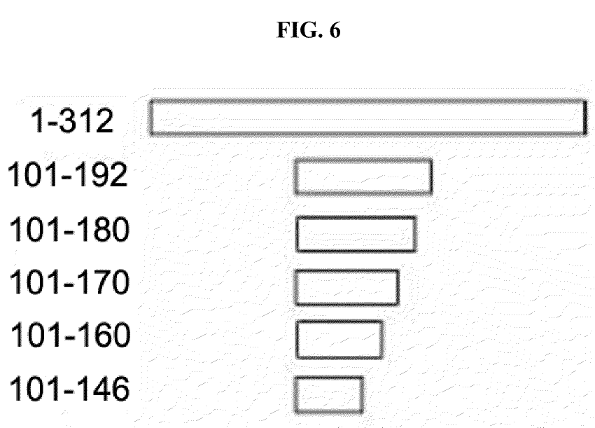

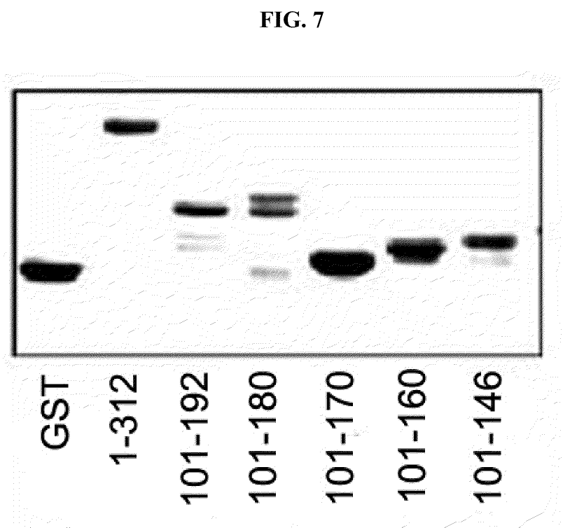

As shown in FIG. 6, the present inventors constructed fragments from the AIMP1-(101-192) by serially deleting C-terminal part of the AIMP1-(101-192) with primers shown in Table.2. Particularly, the same method as in the <Example 1> was performed so as to construct and purify the above fragments. The purified proteins were identified by SDS-PAGE and the results were shown in FIG. 7.

TABLE 2

| Primer | | Sequence | SEQ. ID NO |
|---|---|---|---|
| AIMP1- | sense | 5′-CGGAATTCGCAGTAAC | 35 |
| (101-180) | | AACCGTATCTTCTGG-3′ | |
| | anti- | 5′-GTCTCGAGTTAATCTACTT | 36 |
| | sense | CTTCCACATACAAAGAATC-3′ | |
| AIMP1- | sense | 5′-CGGAATTCGCAGTAAC | 37 |
| (101-170) | | AACCGTATCTTCTGG-3′ | |
| | anti- | 5′-GTCTCGAGTTAATCAGGG | 38 |
| | sense | TGTTTTCTAGCAGTTATG-3′ | |
| AIMP1- | sense | 5′-CGGAATTCGCAGTAAC | 39 |
| (101-160) | | AACCGTATCTTCTGG-3′ | |
| | anti- | 5′-GTCTCGAGTTAACCAAT | 40 |
| | sense | TCGAAGATCCAGACGGG-3′ | |
| AIMP1- | sense | 5′-CGGAATTCGCAGTAAC | 41 |
| (101-146) | | AACCGTATCTTCTGG-3′ | |
| | anti- | 5′-GTCTCGAGTTAGTCGGCA | 42 |
| | sense | CTTCCAGCTATTGATTG-3′ | |

Primer sets used for constructing AIMP1-(101-192) fragments.

As shown in FIG. 7, AIMP1-(101-192) (SEQ ID NO: 7), AIMP1-(101-180) (SEQ ID NO: 8), AIMP1-(101-170) (SEQ ID NO: 9), AIMP1-(101-160) and AIMP1-(101-146) fragments were constructed.
<3-2> Examination of Endothelial cell Death-Inducing Activity of Additional Deletion Fragments of AIMP1-(101-192)

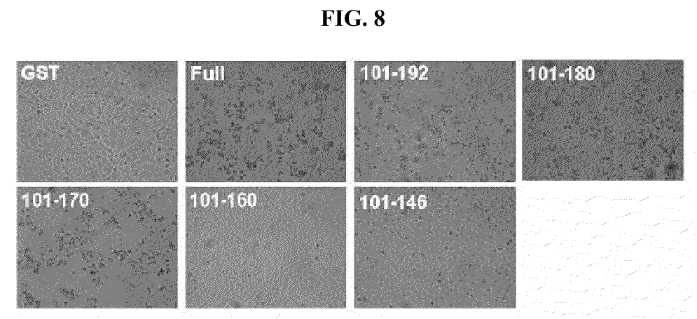

The BAECs cultured by the same method as in the <Example 2-1> were treated with each 50 n of MAIMP1 fragments constructed in the <Example 3-1>, AIMP1 protein and AIMP1-(101-192) fragment constructed in the <Example 1>. The present inventors examined endothelial cell death through morphological change with a light microscope (CX31, OLYMPUS) and the results were shown in FIG. 8.

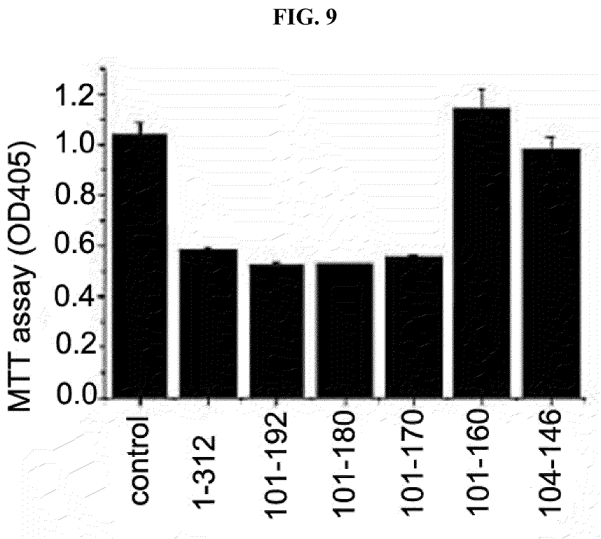

Also, each of 50 nM of AIMP1 fragments constructed in the <Example 3-1>, AIMP1 protein and AIMP1-(101-192) constructed in the <Example 3-1> was treated on the BAECs for 24 hr, respectively. Then MTT solution(5 mg/Ml) was added as much as 1/10 of the volume of cell culture solution and was reacted for 1 hr. Then, after the culture solution was entirely discarded, DMSO (200 μl) was added, absorbance was measured at 570 nm, and the results were shown in FIG. 9.

As shown in FIG. 8 and FIG. 9, it was found that AIMP1 protein, AIMP1-(101-192), AIMP1-(101-180) and AIMP1-(101-170) could induce apoptosis of endothelial cell, but AIMP1-(101-160) and AIMP1-(101-146) could not induce apoptosis. From the result, it was believed that the middle region of AIMP1, especially AIMP1-(101-170), might be a cell death-inducing domain on endothelial cell.

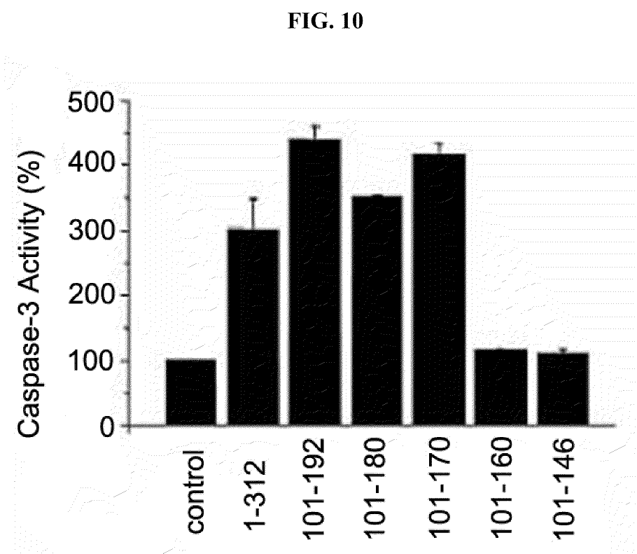

Also, in order to verify the above results, the present inventors measured the effects of the fragments of AIMP1-(101-192) on caspase-3 activity by the same method as in the <Example 2-2>. Then the results were shown in FIG. 10.

As shown in FIG. 8 to FIG. 10, it was found that AIMP1-(1-312) (SEQ. ID NO: 1), AIMP1-(101-192) (SEQ. ID NO: 7), AIMP1-(101-180) (SEQ. ID NO: 8) and AIMP1-(101-170) (SEQ. ID NO: 9) could induce apoptosis of endothelial cell at high levels, but AIMP1-(101-160) and AIMP1-(101-146) could not induce. From the result, it was believed that the middle region of AIMP1, especially AIMP1-(101-170) would be a cell death-inducing domain on endothelial cell.

Consequently, it was found that the AIMP1-(101-170) (SEQ. ID NO: 9) domain would be useful and applicable for anti-angiogenic and anti-tumor therapy.

### Example 4

### <4-1> Construction of AIMP1-(101-170) C161S Protein

In order to construct AIMP1-(101-170) mutant, C161S point mutation in AIMP1-(101-170) polypeptide, which showed cell death inducing activity in the <Example 3>, was generated by PCR. Particularly, the AIMP1-(101-170) mutant was synthesized by PCR using the cDNA of AIMP1 as a template with the following primer set.

forward primer:
(SEQ ID NO: 43)
5′-CGCATATGGCAGTAACAACCGTATCTTCTGGT-3′,
reverse primer:
(SEQ ID NO: 44)
5′-CGCTCGAGTTAATCAGGGTGTTTTCTAGCAGTTATGATGCTACCAA
TTCGAAGATCCAGACGGGA-3′

The PCR reaction conditions were as follows: pre-denaturation of template DNA by heating at 92° C. for 2 min; and then 30 cycles at 92° C. for 30 sec, 56° C. for 30 sec and 72° C. for 20 sec; followed by final extension at 72° C. for 5 min.

After the synthesis, Each of PCR products was digested with NdeI and XhoI and cloned by ligating into a pET49b vector (Novagen) digested with the same restriction enzymes, and AIMP1-(101-170) C161S (SEQ ID NO: 10) was constructed by purifying with the same manner as in <Example 1>.

### <4-2> Analysis of Cell Death Effects of AIMP1 Full Length, AIMP1-(101-170) Polypeptide and AIMP1-(101-170) Mutant

The present inventor found that the AIMP1 full length protein (SEQ. ID NO: 1) or AIMP1-(101-170) polypeptide (SEQ. ID NO: 9) lost endothelial cell death inducing activity in the absence of powerful reducing agent, DTT (Data not shown).

Accordingly, the AIMP1 full length protein (SEQ. ID NO: 1) and AIMP1-(101-170) polypeptide (SEQ. ID NO: 9) constructed in the <Example 1>, were examined about endothelial cell death activities in the presence of DTT, but the AIMP1-(101-170) C161S (SEQ. ID NO: 10) constructed in the <Example 4-1>, in the absence of DTT.

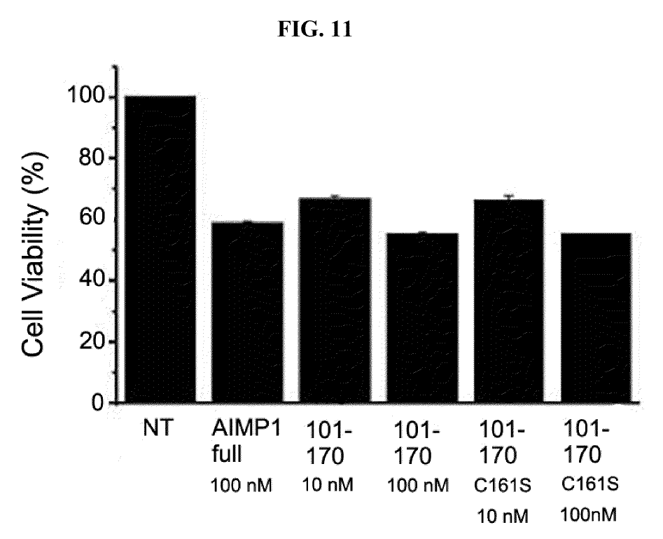

Particularly, the AIMP1 full length protein (100 nM) and AIMP1-(101-170) polypeptide (10, 100 nM) constructed in the <Example 1> were purified in the presence of DTT, but the AIMP1-(101-170) C161S (10, 100 nM) constructed in the <Example 4-1>, in the absence of DTT. Then the purified products were added to BAECs, and the BAECs were cultured for 24 hr. Then MTT assay was performed by the same method as in the <Example 3>, cell viability was measured and the results were shown in FIG. 11.

As shown in FIG. 11, it was found that the AIMP1-(101-170) C161S (SEQ. ID NO: 10) had comparable cell death activity with the AIMP1 full length protein (SEQ. ID NO: 1) or the AIMP1-(101-170) polypeptide (SEQ. ID NO: 9) in spite of the condition of the absence of powerful reducing agent, DTT.

### Example 5

### Anti-Tumor Activity of AIMP1-(101-170) Polypeptide

In order to confirm whether AIMP1-(101-170) polypeptide (SEQ. ID NO: 9) has anti-tumor activity, the present inventors used a xenograft system (BALB/c-nu/nu mouse-MKN-45).

5-week-old male BALB/c-nu/nu nude mice (human tumor xenograft experiments) were obtained from Harlan Co. Ltd. (USA). The mice were housed in a pathogen-free barrier facility with ambient light controlled automatically to produce 12 h light and dark cycles. The MKN-45 (human gastric adenocarcinoma) lines were obtained from the Cell Bank Facility, Korea Research Institute of Biotechnology (KRIBB).

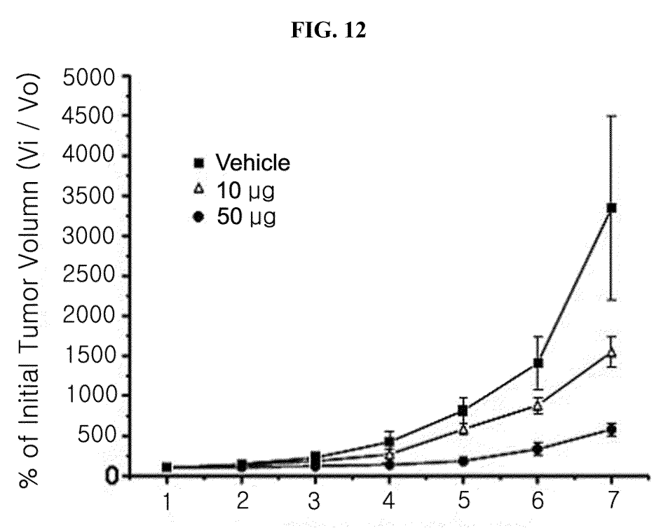

The inventors injected human MKN-45 (gastric cancer) cells adjusted to 2×10⁵ cells/Ml, subcutaneously into the right scapular region of each mouse in a total volume of 50 μl of PBS and monitored the tumor growth. Tumor size was measured in three dimensions with calipers, and tumor volume was calculated as Length×Width×Depth×½. When the tumor size was about 50 mm³ in volume, the mice were administered each of 10 μg/dose and 50 μg/dose of the AIMP1-(101-170) polypeptide constructed in the <Example 3-1> every day for 7 days and relative tumor volume (RTV) was calculated. The relative tumor volume (RTV) was calculated as Vi/Vo, where Vi is the tumor volume at any given time and Vo is the volume at the start of treatment. Based on the result, tumor growth inhibition data were analyzed by student's t-test (p<0.01), and the results were shown in FIG. 12.

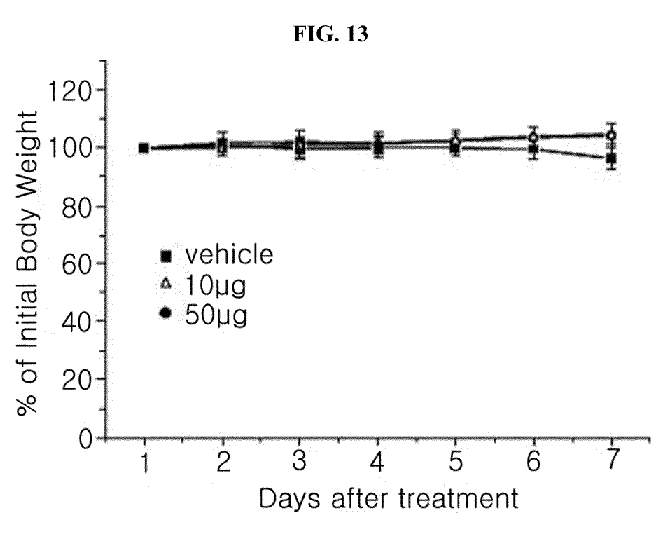

As shown in the FIG. 12, the tumor volume increased about 34 fold in the control groups on the seventh day, whereas it increased only about 15 fold in the 10 μg/dose AIMP1-(101-170) polypeptide group and about 5 fold in the 50 μg/dose AIMP1-(101-170) polypeptide group. And, the present inventors confirmed whether the body weight of the mice was lost. From a result, it was found that treatment of the AIMP1-(101-170) polypeptide did not induce a loss of body weight (see FIG. 13).

On the other hand, the tumors were excised and weighed on day 7. Then tumor growth inhibition rate (TGI) was calculated by the following math expression 1, and the results were shown in the Table 3.

TGI=(1−T/C)×100 [Math Expression 1]

T: the mean final tumor weight of the treated group
C: the mean final tumor weight of the control group

TABLE 3
Anti-tumor activity of AIMP1-(101-170) polypeptide

| | | average tumor | | |
|---|---|---|---|---|
| treatment | dose | weight(g) | TGI(%) | t-test |
| Vehicle | | 5.96 ± 1.64 | | |
| AIMP1-(101-170) | 10 μg/dose | 2.36 ± 0.78 | 60.4 | p = 0.023 |
| polypeptide | | | | |
| AIMP1-(101-170) | 50 μg/dose | 1.30 ± 0.43 | 78.2 | p < 0.01 |
| polypeptide | | | | |

As shown in the Table 3, it was found that the AIMP1-(101-170) polypeptide had anti-tumor activity without inducing a loss of body weight, and would be useful for anti-tumor therapy.

### Example 6

### Comparison of Anti-Tumor Activity Between the AIMP1-(101-170) Polypeptide and the AIMP1 Full Length Protein

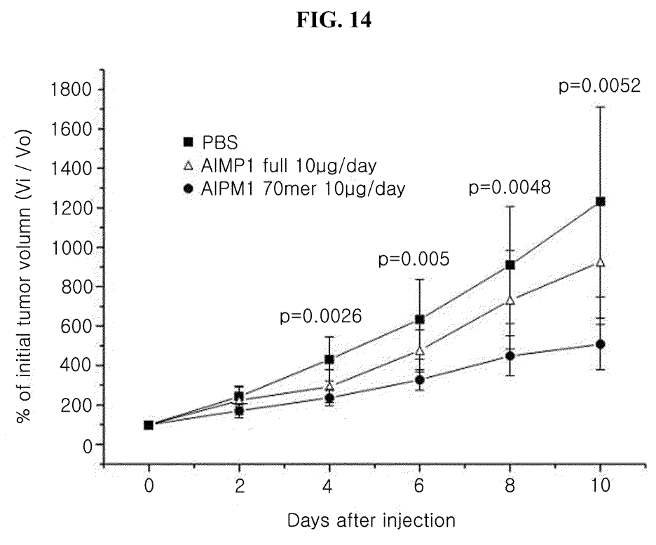

In order to comparison of anti-tumor activity between the AIMP1-(101-170) polypeptide (SEQ. ID NO: 9) and the AIMP1 full length protein, the present inventors performed the same method as in the <Example 5>. But each mouse was administered with 10 μg/dose AIMP1-(101-170) polypeptide and AIMP1 full length protein respectively. Tumor volume was calculated for 10 days and the results were shown in FIG. 14.

The tumor volume increased about 12.3 fold in the vehicle treatment group on day 10 whereas it increased only about 5 fold in the 10 μg/dose AIMP1-(101-170) polypeptide group (p=0.0052) and about 9.2 fold in the 10 μg/dose AIMP1 full length group (p=0.022) (see FIG. 14).

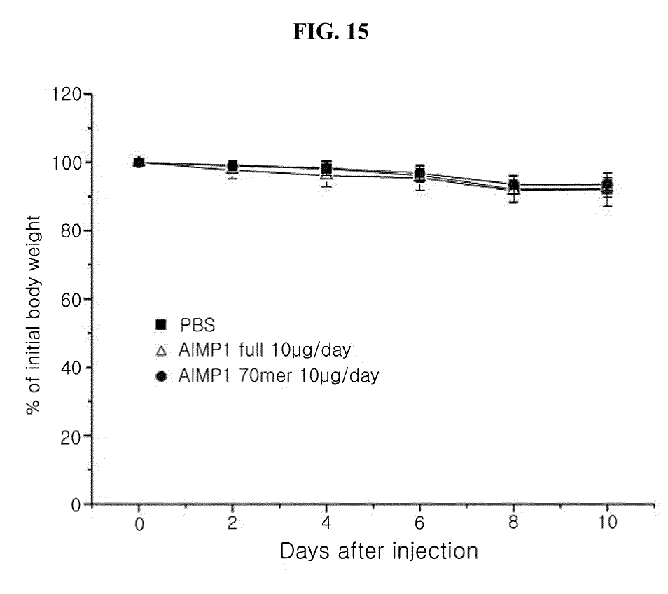

And from the result that the present inventors confirmed whether body weight of the mice was lost, it was found that treating the protein did not induce a loss of body weight in any of the tested animals (see FIG.15). The 10 μg/dose AIMP1-(101-170) polypeptide group showed a 58.75% reduction of tumor volume compared to the control group and the 10 μg/dose AIMP1 full length protein group showed a 24.75% reduction. Accordingly, it was showed that the AIMP1-(101-170) polypeptide had more effective anti-tumor activity than the AIMP1 full length protein.

### INDUSTRIAL APPLICABILITY

The polypeptide or the polynucleotide encoding the polypeptide has an activity of inducing apoptosis of endothelial cell, thus inhibiting cancer cell proliferation. Accordingly, the polypeptide or the polynucleotide encoding the polypeptide can be effectively used for inducing apoptosis of endothelial cell, or preventing or treating cancer.

1. An isolated polypeptide consisting essentially of the amino acid sequence of SEQ ID NO: 9 or an amino acid sequence having at least 90% sequence identity to the amino acid sequence of SEQ ID NO: 9.

2. The isolated polypeptide of claim 1, which consists of an amino acid sequence selected from the group consisting of SEQ ID NO: 8, 9 and 10-14.

3. An isolated polynucleotide that encodes the polypeptide of claim 1.

4. The isolated polynucleotide of claim 3, which has a base sequence selected from the group consisting of SEQ ID NO: 52 to SEQ ID NO: 58.

5. A vector comprising the polynucleotide of claim 3.

6. A transformant transformed with the vector of claim 5.

7. A pharmaceutical composition comprising
(a) an isolated polypeptide consisting essentially of the amino acid sequence of SEQ ID NO: 9 or an amino acid sequence having at least 90% sequence identity to the amino acid sequence of SEQ ID NO: 9; or
(b) an isolated polynucleotide encoding the polypeptide of (a) and a pharmaceutically acceptable carrier.

8. The pharmaceutical composition of claim 7, which is a pharmaceutical composition for treating cancer.

9. A method for inducing apoptosis of an endothelial cell, comprising treating the endothelial cell with an effective amount of (a) an isolated polypeptide consisting essentially of the amino acid sequence of SEQ ID NO: 9 or an amino acid sequence having at least 90% sequence identity to the amino acid sequence of SEQ ID NO: 9; or (b) an isolated polynucleotide encoding the polypeptide of (a).

10. A method for treating cancer, comprising administering to a subject in need thereof an effective amount of (a) an isolated polypeptide consisting essentially the amino acid sequence of SEQ ID NO: 9 or an amino acid sequence having at least 90% sequence identity to the amino acid sequence of SEQ ID NO: 9; or (b) an isolated polynucleotide encoding the polypeptide of (a).

11-13. (canceled)

14. The method of claim 9, wherein the cell is in a subject in need thereof.

15. A fusion protein comprising the polypeptide of claim 1.

16. The fusion protein of claim 15, comprising an expression tag.
